(19) United States
(12) Patent Application Publication
(10) Pub. No.: US 2020/0098458 A1
(43) Pub. Date: Mar. 26, 2020

(54) MEDICAL CANNABIS PLATFORM WITH PHYSICIAN AND PATIENT PORTALS

(71) Applicant: Arfinn Learning Solutions, Inc., Dallas, TX (US)

(72) Inventors: James Andersen WEST, Dallas, TX (US); Jack Edgar WEST, Tampa, FL (US)

(21) Appl. No.: 16/581,980

(22) Filed: Sep. 25, 2019

Related U.S. Application Data
(60) Provisional application No. 62/737,016, filed on Sep. 26, 2018.

(51) Int. Cl. G16H 20/10; G16H 80/00; A61K 36/185

(52) U.S. Cl. CPC G16H 20/10; A61K 36/185; G16H 80/00

(57) ABSTRACT

Through a physician's portal, a platform calculates efficacy ratings for patient records matching a medical condition and generates reports. The physician uses the reports to create a cannabis treatment prescription for the patient, which is stored in the patient's record. An electronic notification message is sent to the patient with a unique access code, which the patient may use to retrieve the cannabis treatment prescription via a patient's portal. After the patient obtains cannabis products, the patient inputs cannabis treatment data via the patient's portal, including cannabis profile of the cannabis products and a patient efficacy rating, which are stored in the patient's record. The physician retrieves the cannabis treatment data via the physician's portal and optionally modifies the prescription. The patient retrieves the modified prescription via the patient's portal. In this manner, networking communication channels between physician and patient for cannabis treatment and result data are provided.

### BACKGROUND OF THE INVENTION

Alternative and or non-traditional medicines, such as medical cannabis, are becoming increasingly important to people seeking treatment for various medical conditions. One of the challenges for medical professionals is acquiring credible data of effective treatments, efficacies and best practices for this emerging medical industry. For example, an increasing number of physicians today are faced with patients who are considering medical cannabis as a viable option of treatment, yet that knowledge is fragmented and often anecdotal. In many instances this lack of credible efficacy data inhibits physicians from recommending cannabis to patients, or only recommending certain treatments with which they experience through trial and error.

When patients obtain cannabis products from cannabis dispensaries, the dispensaries are often not obligated to dispense only the cannabis product prescribed by the physician. The actual cannabis product received by the patient is typically agreed upon between the dispensary and the patient without the involvement of the physician. Another challenge for medical professionals is the difficulty in being informed of the cannabis product used by the patient and/or receiving feedback from the patient on the results from the cannabis treatment. This information would not typically be disclosed to the physician until a follow-up appointment with the patient, which, in some jurisdictions, is not required until a patient needs to be re-certified for medical cannabis.

### BRIEF SUMMARY OF THE INVENTION

Disclosed herein is a medical cannabis platform with physician and patient portals, and a corresponding system and a computer program product as specified in the independent claims. Embodiments of the present invention are given in the dependent claims. Embodiments of the present invention can be freely combined with each other if they are not mutually exclusive.

According to one embodiment of the present invention, a computing platform provides networking communication channels between a physician and a patient for cannabis treatment and result data. The computing platform receives input of a medical condition for a patient from a first client device of a physician. The platform finds a set of patient records in a database matching the medical condition and calculates an efficacy rating for the set of patient records. The platform then generates, for display at the first client device, report(s) of cannabis treatment profiles in the set of patient records and corresponding efficacy ratings. When the platform receives from the first client device a cannabis treatment prescription for the patient, the platform stores the cannabis treatment prescription in a patient record:associated with the patient. The platform then generates a unique access code, associates the unique access code with the patient, and sends an electronic message to the patient that includes the unique access code and optionally a notification of an availability of the cannabis treatment prescription for the patient.

In one aspect of the present invention, the platform receives modification(s) to a given cannabis treatment profile in the report(s) and receives a selection of the modified given cannabis treatment profile as the cannabis treatment prescription for the patient from the first client device.

In one aspect of the present invention, the platform receives an access request that includes the unique access code from a second client device of the patient. In response, the platform authenticates access by the second client device using the unique access code. When the platform receives a request from the second client device for the cannabis treatment prescription for the patient, the platform retrieves and outputs the cannabis treatment prescription stored in the patient record associated with the patient.

In one aspect of the present invention, after the patient obtains cannabis product(s), the platform receives input of cannabis treatment data for the patient from the second client device. The input includes a cannabis profile of the cannabis product(s) and a patient efficacy rating. The platform stores the cannabis treatment data in the patient record associated with the patient and'sends an electronic message to the physician that includes a notification of an availability of the cannabis treatment data input by the patient.

In one aspect of the present invention, the platform receives a request for the cannabis treatment data input by the patient from the first client device. In response, the platform retrieves the patient record associated with the patient from the database.

In one aspect of the present invention, the platform receives modification(s) to the cannabis treatment prescription for the patient from the first client device. In response, the platform stores the modified cannabis treatment prescription in the patient record associated with the patient and sends an electronic message to the patient that includes a notification of an availability of the modified cannabis treatment prescription.

### BRIEF DESCRIPTION OF THE SEVERAL VIEWS OF THE FIGURES

FIG. 1 illustrates an exemplary computing environment providing the cannabis treatment platform according to some embodiments of the present invention.

FIG. 2 illustrates an exemplary cannabis treatment platform accessible through a physician portal according to some embodiments of the present invention.

FIG. 3 illustrates an exemplary method for calculating the efficacy ratings and the generation of the reports according to some embodiments of the present invention.

FIG. 4 illustrates an exemplary cannabis treatment platform accessible through a patient portal according to some embodiments of the present invention.

FIG. 5 illustrates a further exemplary cannabis treatment platform accessible through the physician portal according to some embodiments of the present invention.

FIGS. 6A-6G illustrate example web forms for receiving patient data input.

FIG. 7 illustrates an example web form for inputting report filters.

FIGS. 8A-8C illustrates example reports generated by the platform based on average efficacy ratings or frequency distribution of efficacy ratings.

FIG. 9A illustrates an example patient portal login interface.

FIG. 9B illustrates an example of a patient portal input web form.

FIG. 10 illustrates a computing system, one or more of which may be used to implement the server, the patient client device, and/or the physician client device, according to various embodiments of the present invention.

### DETAILED DESCRIPTION OF THE INVENTION

The following description is presented to enable one of ordinary skill in the art to make and use the present invention and is provided in the context of a patent application and its requirements. Various modifications to the embodiment will be readily apparent to those skilled in the art and the generic principles herein may be applied to other embodiments. Thus the present invention is not intended to be limited to the embodiment shown but is to be accorded the widest scope consistent with the principles and features described herein.

Reference in this specification to “one embodiment”, “an embodiment”, “an exemplary embodiment”, or “a preferred embodiment” means that a particular feature, structure, or characteristic described in connection with the embodiment is included in at least, one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments. In general, features described in one embodiment might be suitable for use in other embodiments as would be apparent to those skilled in the art.

Embodiments of the present invention provide a cannabis treatment platform that includes a physician portal and a patient portal. The platform advances the knowledge and effective use of medical cannabis treatments and provides factual peer-to-peer cannabis efficacy outcomes for physicians and patients with statistical inference capability, allowing them to search for the most effective cannabis treatment based on medical condition and optionally demographic information. Physicians and patients thus will be able to make more informed decisions regarding treatment. The platform further provides network communication channels between physicians and patients for the sharing of cannabis treatment prescriptions and result data, allowing physicians to further tailor the recommended cannabis treatments to the patient.

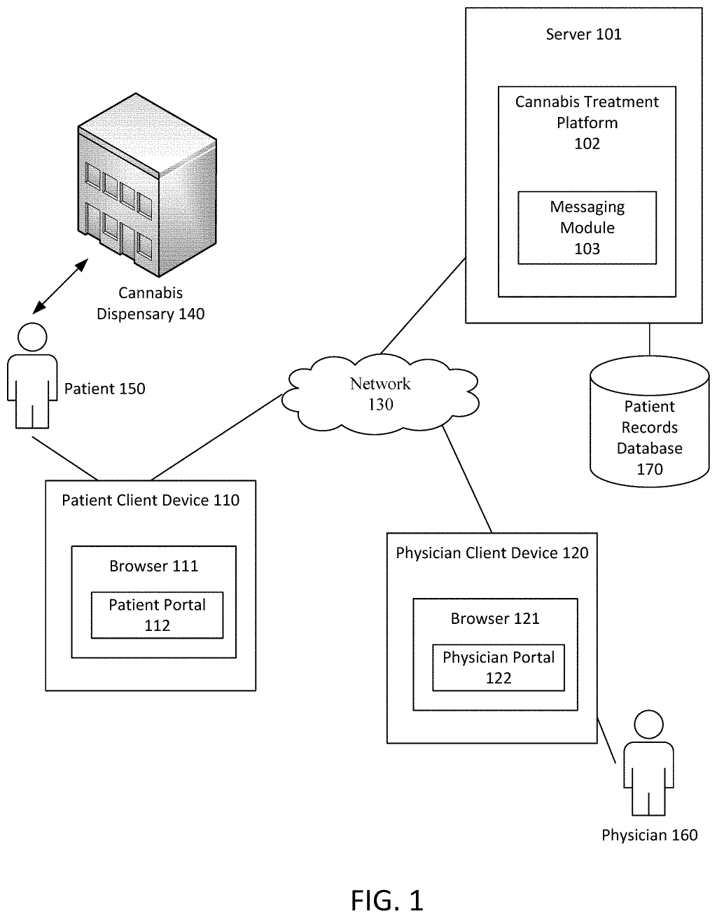

FIG. 1 illustrates an exemplary computing environment providing the cannabis treatment platform according to some embodiments of the present invention. A server 101 provides the cannabis treatment platform 102 which includes a messaging module 103. In some embodiments, the server 101 is an application server hosting one or more web applications, which when executed by the server 101, implements the cannabis treatment platform as described herein. The cannabis treatment platform 102, via the messaging module 103, is configured with the ability to send and receive electronic messages (e.g. SMS, MSS, email, message boards, and online forums) to a patient 150 vis a patient client device 110 and/or to a physician 160 via a physician client device 120. The server 101 is configured with access to a patient records database 170, where each patient record stores patient data associated with a particular patient. The patient 150 accesses and navigates the cannabis treatment platform 102 over a network 130 (e.g. the Internet) through a patient portal 112 implemented by a browser 111 on the patient client device 110. The physician 160 accesses and navigates the cannabis treatment platform 102 over the network 130 through a physician portal 122 implemented by a browser 121 at the physician client device 120. The patient 150 physically, or via an online portal, visits a cannabis dispensary 140 to purchase cannabis products. In some embodiments, the cannabis treatment platform 102 is configured to be restricted to licensed medical professionals, and cannabis dispensaries 140 are not configured with access to the cannabis treatment platform 102.

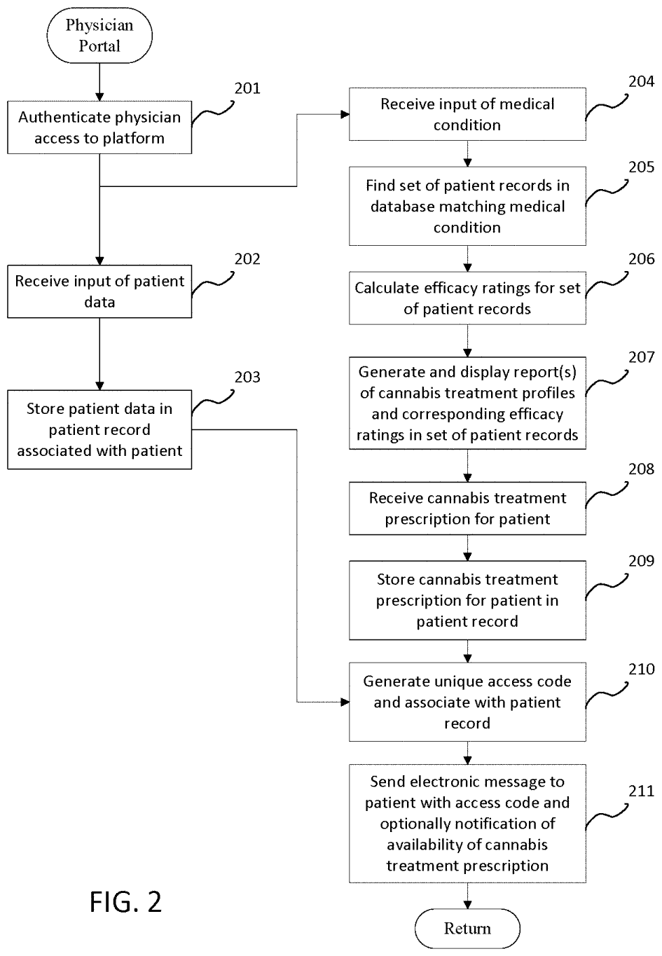

FIG. 2 illustrates an exemplary cannabis treatment platform accessible through a physician portal according to some embodiments of the present invention. Access to the platform 102 122 is configured;to be restricted to licensed medical professionals. As used herein, the “physician” 160 includes persons legally licensed to practice medicine by a medical licensing body in the person's jurisdiction. Optionally, other medical professionals with a license to prescribe treatment can be included as a “physician” user 160, such as physician assistants and registered nurses. Optionally, users otherwise authorized to recommend cannabis treatments can be a “physician” user 160. To access the cannabis treatment platform 102, the physician 160 first registers with the platform 102. The physician 160 provides the platform 102 with identifying information, including licensing information. The platform 102 validates the identity and credentials of the physician, after which the physician 160 is configured with unique login credentials for accessing the platform 102. Optionally, the platform additionally validates that the physician 160 has a license in a jurisdiction where medical cannabis is legal. The physician 160 provides the login credentials to the platform 102 via the physician portal 122, and once the login credentials are authenticated (201), is given access to the cannabis treatment platform 102.

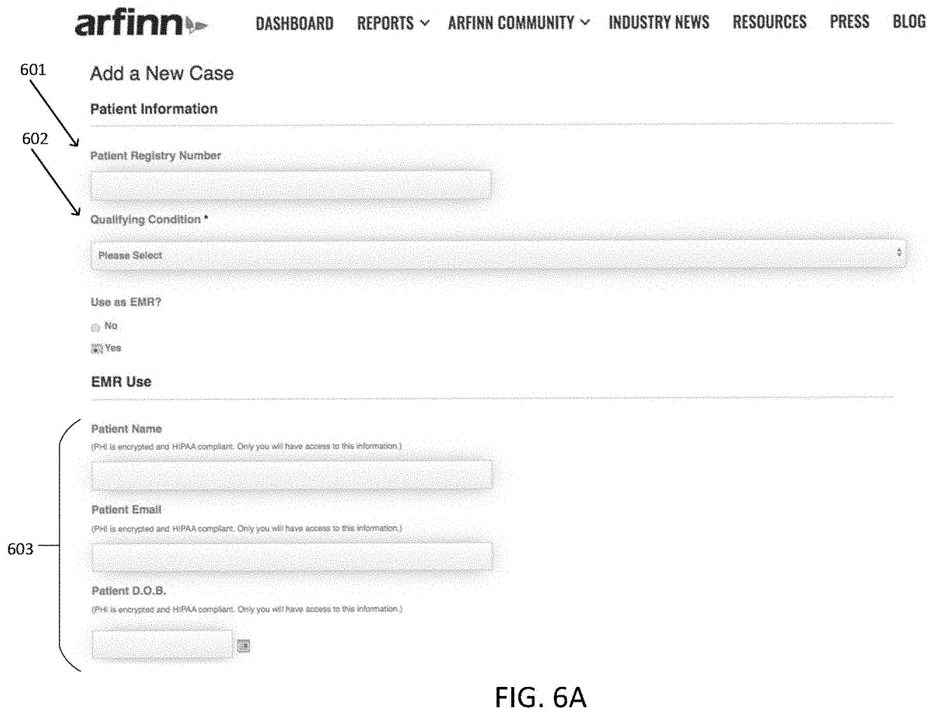

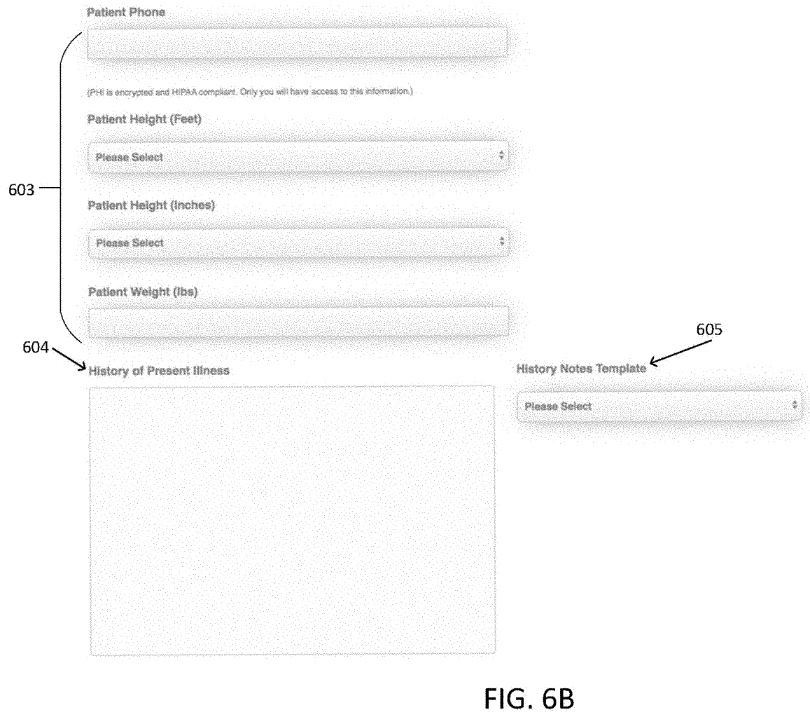

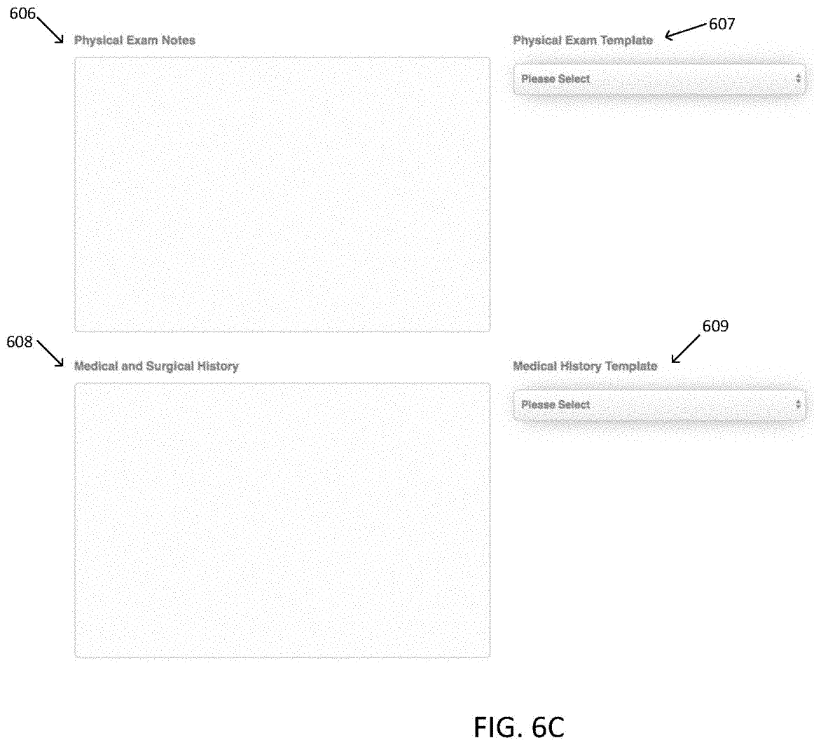

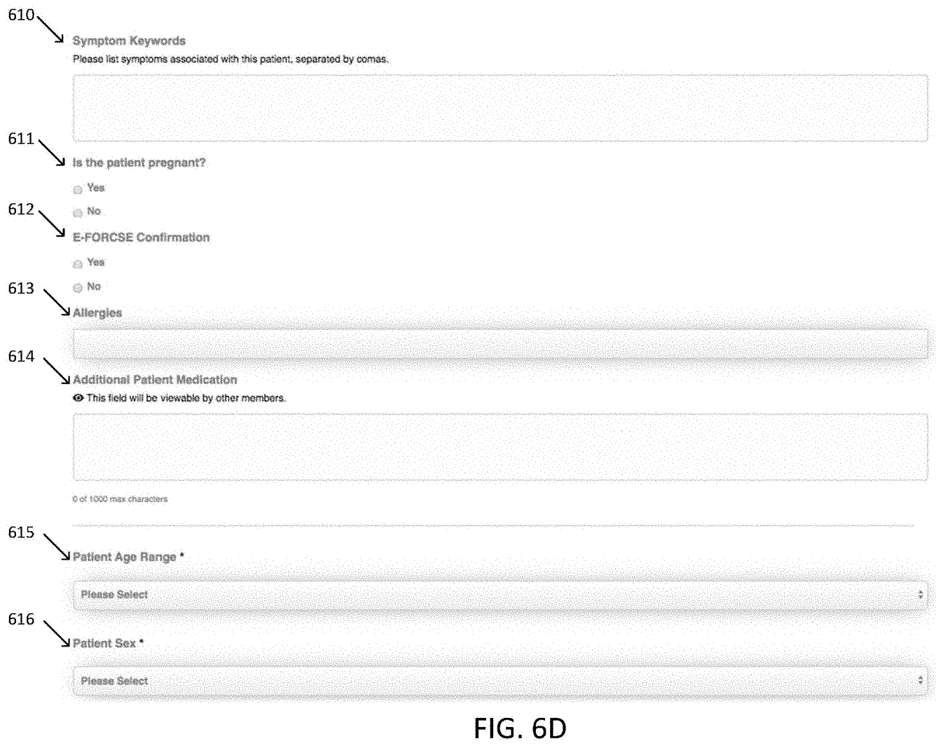

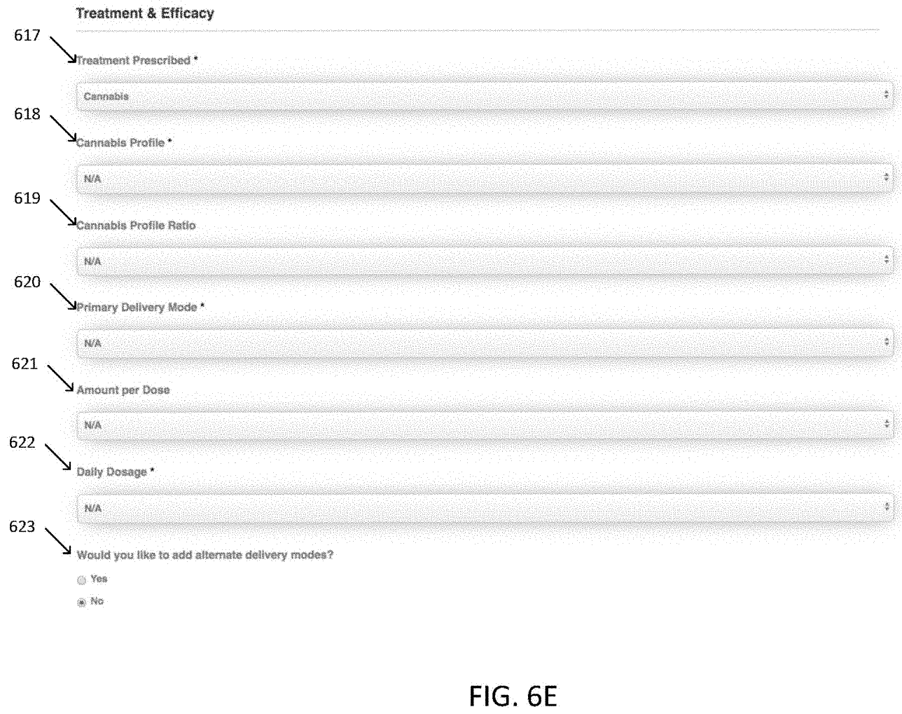

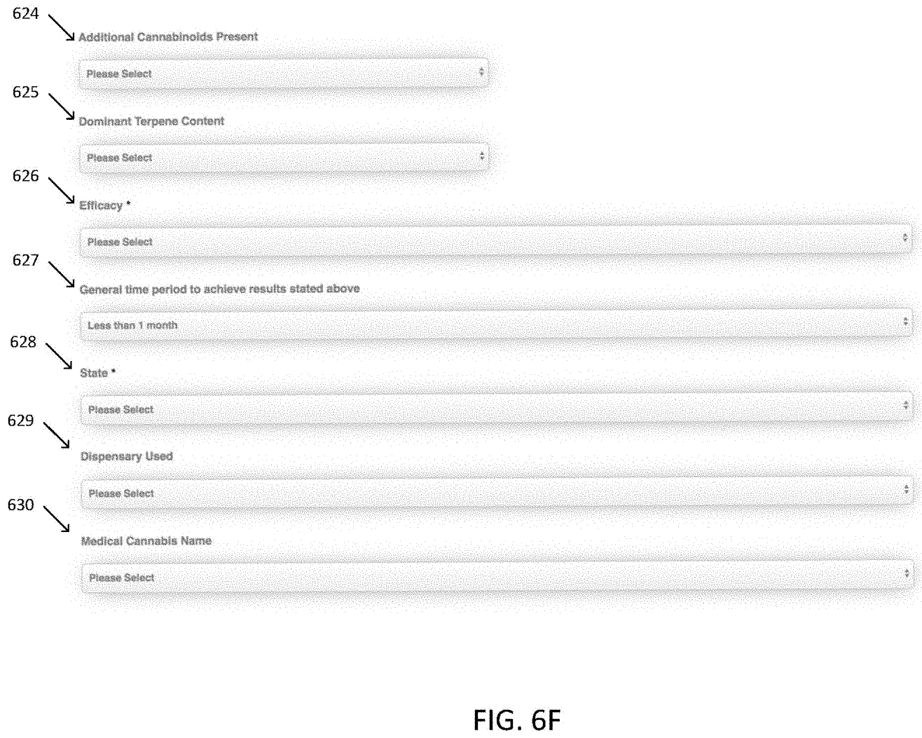

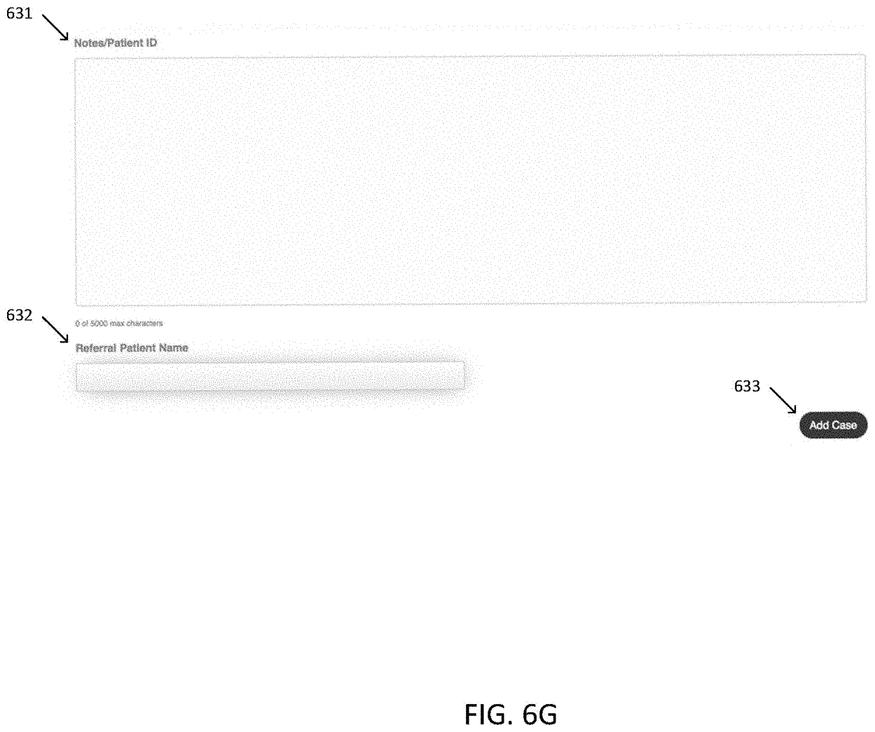

The physician's access to and navigation through the platform 102 is facilitated by the physician portal 122. Through the physician portal 122, the physician 160 can choose to input patient data and/or generate reports regarding potential cannabis treatments for the patient 150. The physician 160 selects the desired function through the physician portal 122. In some embodiments, when the physician 160 selects the patient data input function, the physician portal 122 provides a web form into which the physician 160 enters the patient data. FIGS. 6A-6G illustrate example web forms for receiving patient data input. Each patient is assigned a unique patient registry number 601. As illustrated in FIG. 6A, the physician 160 identifies the patient 150 by entering the patient's registry number 601 and inputs the patient's qualifying condition 602. The patient's registry number 601 is a number assigned to the patient 150 by a jurisdiction once the patient 150 is deemed by a physician to have a condition which qualifies the patient 150 to receive medical cannabis, referred herein as a “qualifying condition”. As illustrated in FIGS. 6A and 6B, the physician 160 may also input certain patient information 603 for electronic medical records (EMR) use, such as the patient name, email, date of birth, phone, height, and weight.

As illustrated in FIGS. 6B and 6C, the physician 160 inputs: a history of the patient's present illness 604, optionally using a history notes template 605; physical exam notes 606, optionally using a physical exam template 607; and the patient's medical and surgical history 608, optionally using a medical history template 609.

As illustrated in FIG. 6D, other physician 160 inputs may include: the patient's symptoms using keywords 610; whether the patient is pregnant 611; whether the patient is part of a prescription monitoring database, such as Prescription Monitoring Program (PMP) Gateway or the Electronic-Florida Online Reporting of Controlled Substance Evaluation Program (E-FORCSE) 612; any allergies 613; any additional patient medication 614; patient age range 615; and patient sex or gender 616.

As illustrated in FIGS. 6E-6F, the physician 160 further inputs the prescribed cannabis treatment and the efficacy data for the treatment, which may include: the treatment prescribed 617; the cannabis profile 618, the cannabis profile ratio 619 of THC to CBD; the primary delivery mode 620 (e.g. vape pen, tincture, etc.); the amount per dose 621; and the daily dosage 622. For the treatment prescribed, the physician 160 inputs any alternate delivery modes 623, any additional cannabinoids present 624 other THC and CBD, the dominant terpene content 625, the efficacy rating 626 for the prescribed treatment, and the general time period within which to achieve the results 627, and the state where the patient 150 is seeking treatment 628. The physician 160 further inputs the dispensary used 629, if known, and the name of the medical cannabis 630 purchased from the dispensary.

As illustrated in FIG. 6G, a text field 631 is provided for the physician 160 to enter any additional notes. Optionally, the physician 160 can record the name of another patient 632 who referred the patient 150. Once the platform 102 receives the selection of the “Add. Case” button 633 by the physician 160, the platform 102 stores the patient data inputted through the web form(s) in the patient record associated with the patient 150.

The web forms disclosed in FIGS. 6A-6G are for illustrative purposes, and any combination of data and/or additional data may be input through the web forms without departing from the spirit and scope of the present invention.

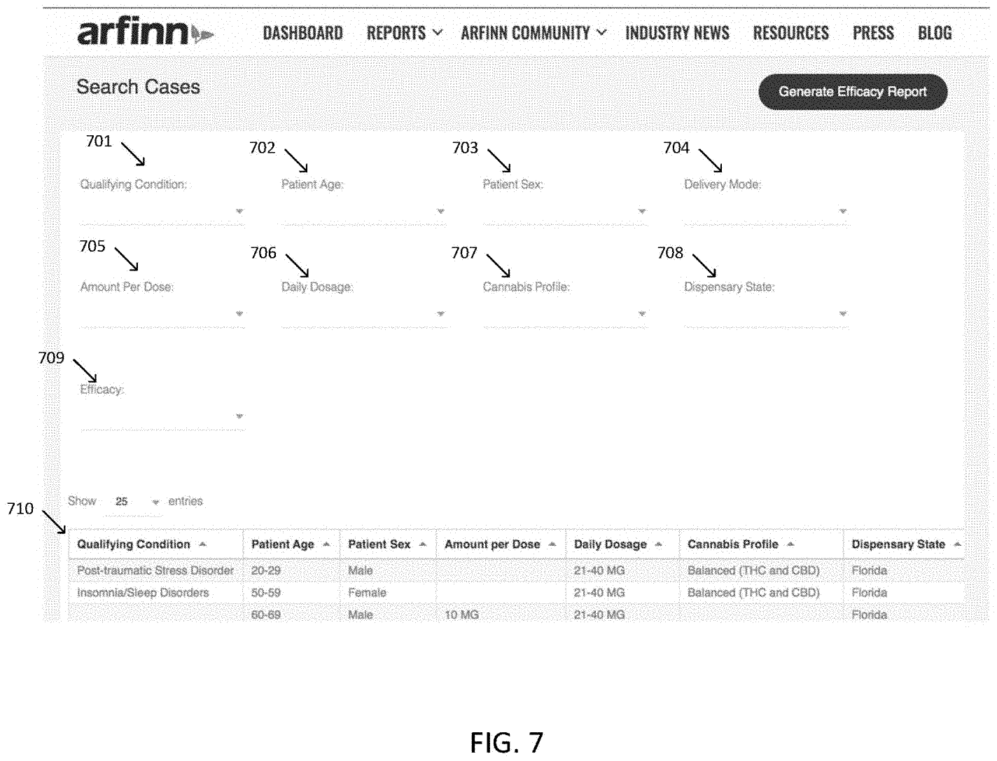

Returning to FIG. 2 when the platform 102 receives the input of patient data from the physician 160 through the physician portal 122 (202), the platform 102 stores the patient data in the patient, record associated with the patient 150 (203). When the physician 160 selects the report generation function, the physician portal 122 provides a web form into which the physician 160 selects or inputs one or more filters for the report. FIG. 7 illustrates an example web form for inputting report filters. In this example, the possible filters include the qualifying or primary medical condition 701 of the patient 150. The filters can include one or more demographic traits of the patient 150, such as the patient's age 702 and sex or gender 703. Other possible filters include parameters for the treatment, such as the delivery mode 704, the amount per dose 705, the daily dosage 706, the cannabis profile 707, the dispensary state 708, and the efficacy rating 709. As the filters are selected, a list 710 of the patient records matching the filters are displayed. Other filters are possible, including some combination of the data as illustrated in FIGS. 6A-6G.

Returning to FIG. 2, when the platform 102 receives the input of the medical condition (204), the platform 102 finds a set of one or more patient records in the database 170 matching the medical condition (205). When additional filters are input, such as demographic traits, the matching patient records are further filtered to exclude the patient records that do not match the demographic traits. The platform 102 then calculates the efficacy ratings for the set of patient records (206). The efficacy ratings represent the effectiveness of the cannabis treatment profiles in the set, of patient records for the medical condition and other filters, if any. The platform 102 generates and displays one or more reports of the cannabis treatment profiles in the set of patient records and their corresponding efficacy ratings (207).

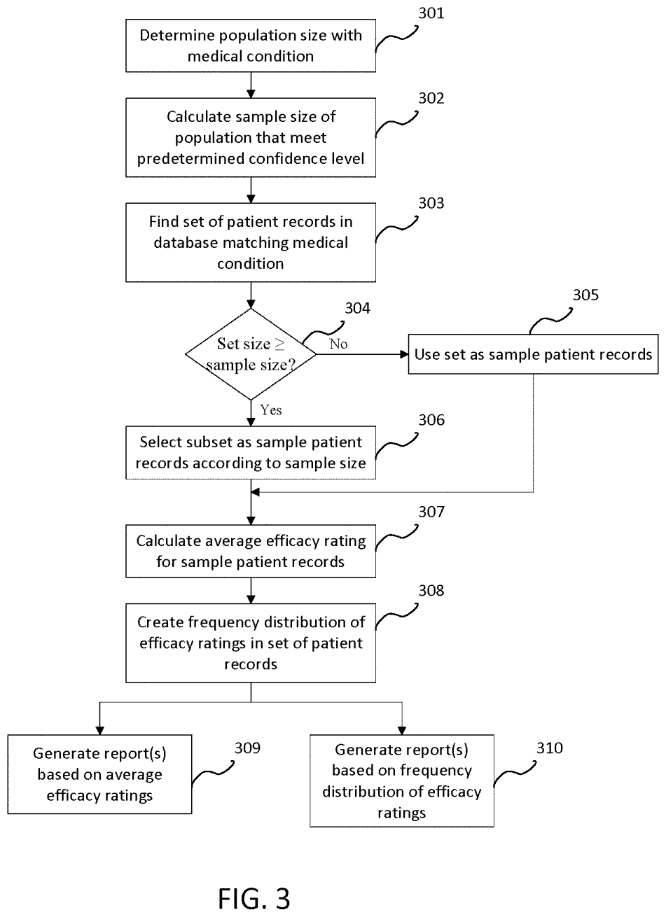

FIG. 3 illustrates an exemplary method for calculating the efficacy ratings and the generation of the reports according to some embodiments of the present invention. Upon receiving the input of the patient medical data, including medical condition (see 204, FIG. 2), the platform 102 determines a population size with the medical condition (301). Using statistical processes, such as multiple regression analysis, the platform 102 calculates a sample size of the population that meets a predetermined confidence interval level (302). When additional filters are input, such as a geographic area for the patient, the population size and sample size is further determined based on the additional filters. In some embodiments, the medical condition is defined as the primary medical condition within a population, gathered from a credible source, such as the National Institute of Health (NIH) for the US. The confidence interval represents the level of confidence that a mean of the efficacy ratings in the sample can be inferred upon the population as, a whole, within a predetermined margin of error. In statistical modeling, regression analysis is a set, of statistical processes for estimating relationships among variables. Here, regression, analysis estimates the relationships among the various patient data in the patient records. The platform 102 finds a set of patient records in the database 170 matching the medical condition, and any other filters (303), and determines whether the number of patient records in the set is greater than or equal to the sample size (304). When the >number of patient records in the set is less than the calculated sample size, the patients records in the set is used as the sample (305). Otherwise, the platform 102 selects a subset of the set as the sample patient records according to the sample size (306). In some embodiments, the subset is randomly selected from the set. The platform 102 then calculates the mean efficacy rating for the sample patient records (307). The average can be any combination of mean, mode, median, or range of efficacy ratings. This calculated mean efficacy rating can be inferred upon the population at the predetermined confidence level and within a predetermined margin of error. The platform 102 further creates a frequency distribution of efficacy ratings in the set of patient records (308). The platform 102 can then generate report(s) based on the average efficacy ratings (309) and/or on, the frequency distribution of efficacy ratings (310), as described further below.

For example, assume that the patient 150 is female, aged 53, resides in the US, and suffering from Parkinson's disease. The platform 102 receives input of Parkinson's disease as the primary medical condition of the patient 150 with the US as an additional geographic filter, along with any other additional filters relevant to the specificity input for the patient by the physician (204). The platform 102, using authoritative medical sources, such as the National Institute of Health (NIH), determines that the population matching the filters is 6000 (301). Assume for this example that the sample size of 381 is calculated for a predetermined 95% confidence level and a predetermined 10% margin of error (302). Assume that the platform 102 finds 750 patient records in the database 170 matching the filters (303). Since the set of matching patient records in the database 170 is greater than the calculated sample size of 381 (304), the platform 102 randomly selects 381 of the patient records from the set of 750 matching patient records (306). The platform 102 then calculates the average efficacy rating for the 381 sample patient records (307). Thus, an efficacy rating within 10% of 3.5 (3.15−3.85) calculated for the sample can be inferred upon the population with Parkinson's (and any other filters) as a whole with a 95% confidence level. The platform 102 also creates the frequency distribution of the efficacy ratings in the 750 matching patient records (308).

In another example, assume that only 250 patient records in the database 170 match the filters (303). Since the set of matching patient records is less than the calculated sample size of 381 (304), all of the 250 patient records in the set is used as the sample patient records (305). The platform 102 then calculates the mean efficacy rating for the 250 sample patient records (307) and creates the frequency distribution of the efficacy ratings for the 250 matching patient records (308).

The platform 102 generates one or more reports, depending on which report is requested by the physician 160 through the physician portal 122, and depending on the specificity filters requested by the physician 160. For example, the physician 160 can submit a request to the platform 102 for a report on efficacy data of medical cannabis for Parkinson's disease in general or for females with Parkinson's disease in a particular age set, along with any other filters such as high blood pressure. Example filters include, but are not limited to: geographic location; dispensary used; dosage; and strength and makeup of the cannabis product used.

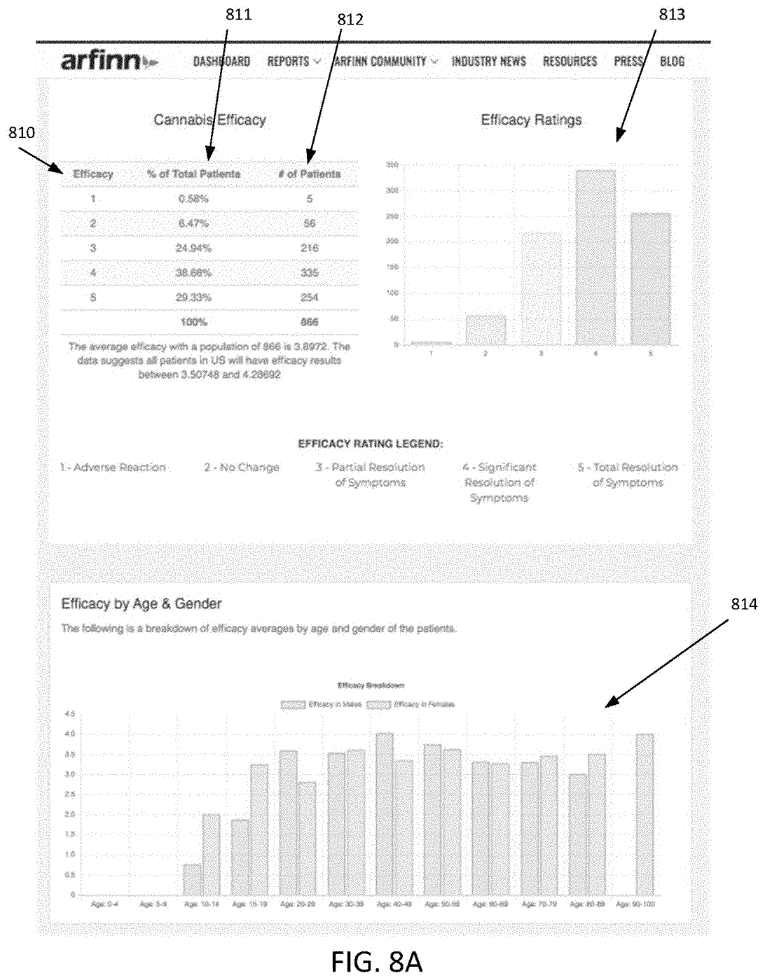

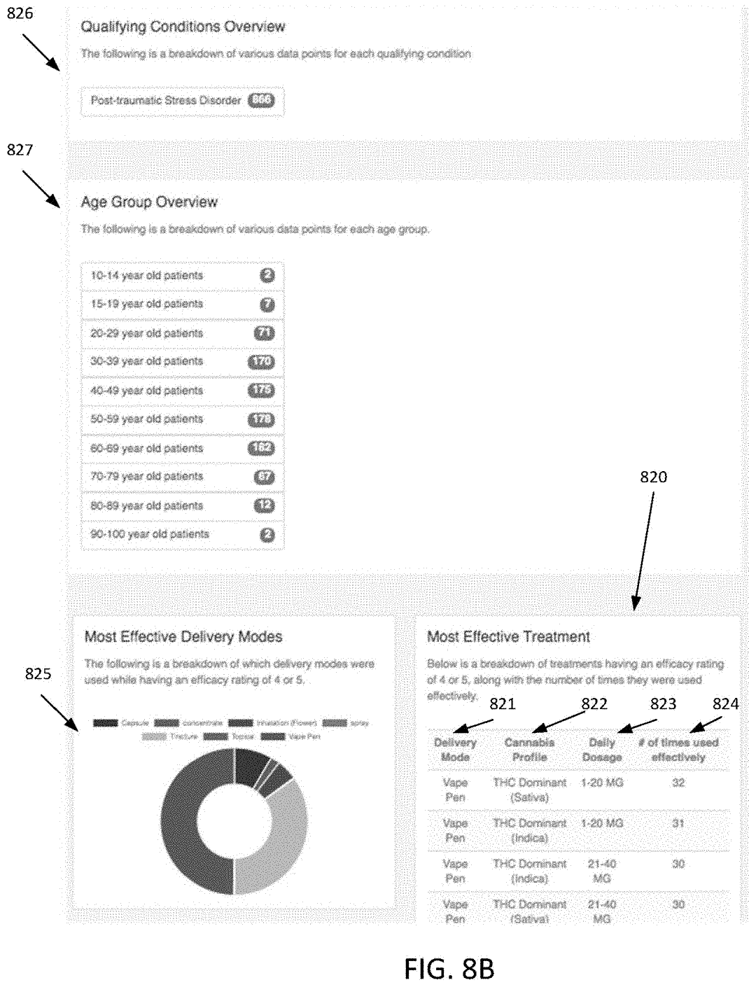

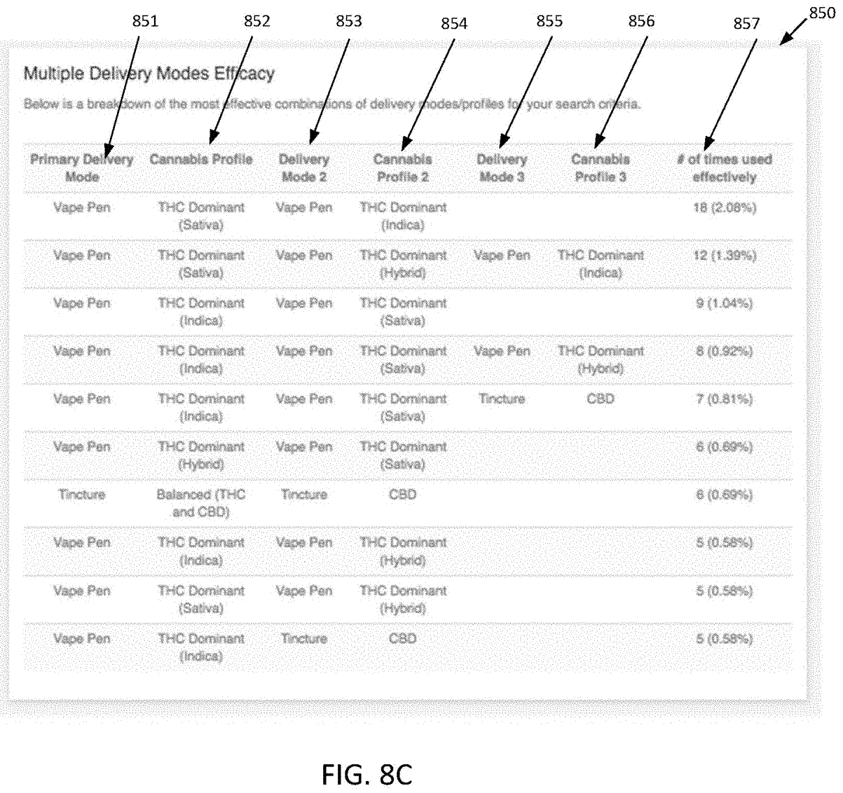

FIGS. 8A-8C illustrates example reports generated by the platform 102 based on average efficacy ratings or frequency distribution of efficacy ratings. As illustrated in FIG. 8A, the example reports can include the average efficacy rating of 3.8972 for a set of patient records numbering 866, calculated per block 307 of FIG. 3. Based on the predetermined margin of error, a range of efficacy ratings of between 3.50748 and 4.28692 is determined. Using the frequency distribution created per block 308 of FIG. 3, the report further includes a breakdown of efficacy ratings 810, the percentage of patient records in the set with each efficacy rating 811, and the number of patient records in the set with each efficacy rating 812. The breakdown can also be displayed in a graphical manner, such as with a bar graph 813. A graphical representation 814 with more granular breakdowns, such as by age and gender of the patient records in the set, can also be generated.

The reports can also provide a breakdown of various data points for each qualifying or primary condition. As illustrated in FIG. 8B, the physician 160 can select the qualifying condition 826, such as post-traumatic stress disorder, of which there are 866 patient records in the set. When a breakdown based on age is requested, the report includes the number of patient records in the set within each age group 827. The reports can further provide a breakdown of delivery modes 825 and which were used while having certain efficacy ratings. The reports can further provide a breakdown of effective treatments 820 having certain efficacy ratings, along with the number of times they were used effectively among the set of patient records. The number of times a treatment was used effectively is the number of times the treatment reached the maximum improvement. In the illustrated example, for an efficacy rating scale of 1-5, with 5 being the most effective, a threshold representing the maximum improvement can be preset to an efficacy rating of 4.0. The report, of effective treatments 720 can include a list of cannabis treatment profiles that meet or exceed a predetermined threshold. The list can include details of the profiles, such as the delivery mode 721, the cannabis profile 722, the daily dosage 723, and the number of times the treatment was used effectively 724, i.e., with an efficacy rating meeting or exceeding the threshold of 4.0. Reports other than those discussed above can also be implemented by the platform 102.

As illustrated in FIG. 8C, the reports can further provide a breakdown 850 of the most effective combinations of delivery modes or profiles for the set of patient records. The breakdown 850 can include different combinations of primary delivery modes 851 and a primary cannabis profile 852, a secondary delivery mode 853 and a secondary cannabis profile 854, and a tertiary delivery mode 855 and a tertiary cannabis profile 856. For each combination, the report includes the number of times the combination was used effectively 857, i.e., had an efficacy rating that meet or exceed the threshold.

Returning to FIG. 2, the physician 160 reviews the reports and uses the report information to determine an appropriate cannabis treatment for the patient 150. The platform 102 receives from the physician an input of a prescribed cannabis treatment for the patient (208). In some embodiments, the platform 102 receives a selection by the physician 160 of a cannabis treatment profile displayed in a report as the prescription for the patient 150. In some embodiments, the physician 160 customizes the cannabis treatment prescription by modifying one or more parameters of the treatment profile, such as mode of delivery or dose. In some embodiments, the physician 160 manually enters a cannabis treatment prescription for the patient 150. The cannabis treatment prescription for the patient 150 is then stored in the patent record associated with the patient 150 (209). The platform 102 then generates a unique access code and associates the access code with the patient record (210). The access code is used by the patient 150 to access the platform 102 via the patient portal 112 in order to input cannabis treatment data, as described further below. In some embodiments, the physician 160 prints the cannabis treatment prescription and delivers it to the patient 150. Alternatively, the cannabis treatment prescription is delivered to the patient electronically by the platform 102, as described further below The platform 102, via the messaging module 103, sends an electronic message to the patient 150 that includes the access code and optionally a notification of the availability of the cannabis treatment prescription (211). In some embodiments, the platform 102 retrieves the contact information from the patient's record and uses the contact information to send a secure electronic message. The secure electronic message may be by, a short messaging service (SMS), a multimedia messaging service (MSS), an E-Mail service, or any other appropriately secure electronic messaging mechanism. The electronic message further includes a request for the patient 150 to enter patient treatment data using the access code in order to capture the effectiveness of the treatment.

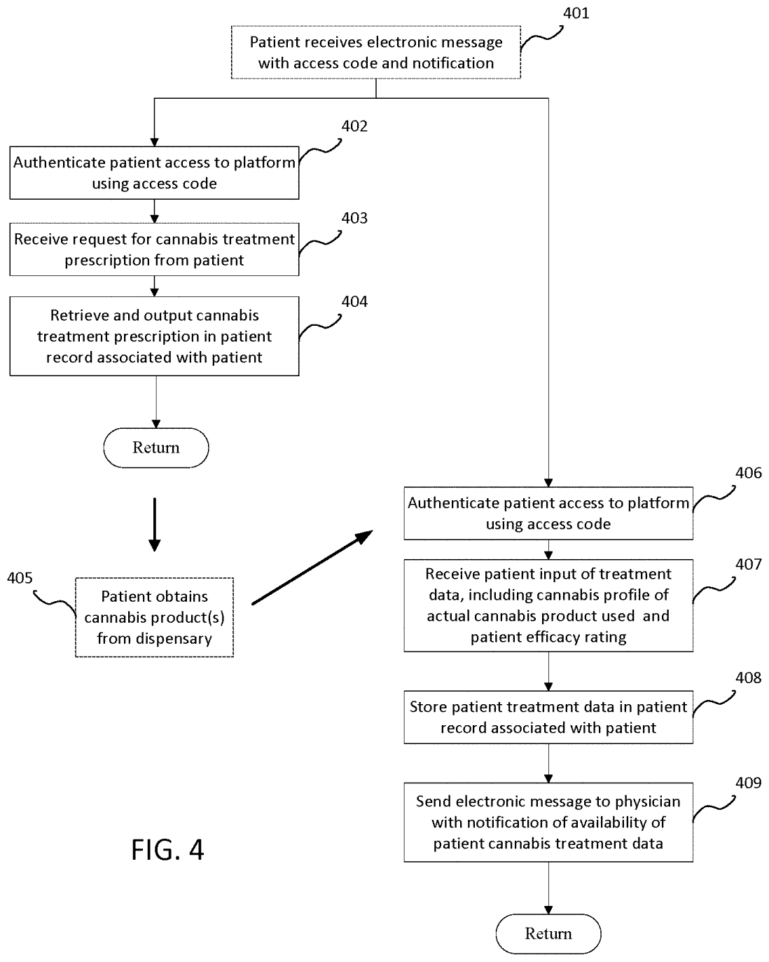

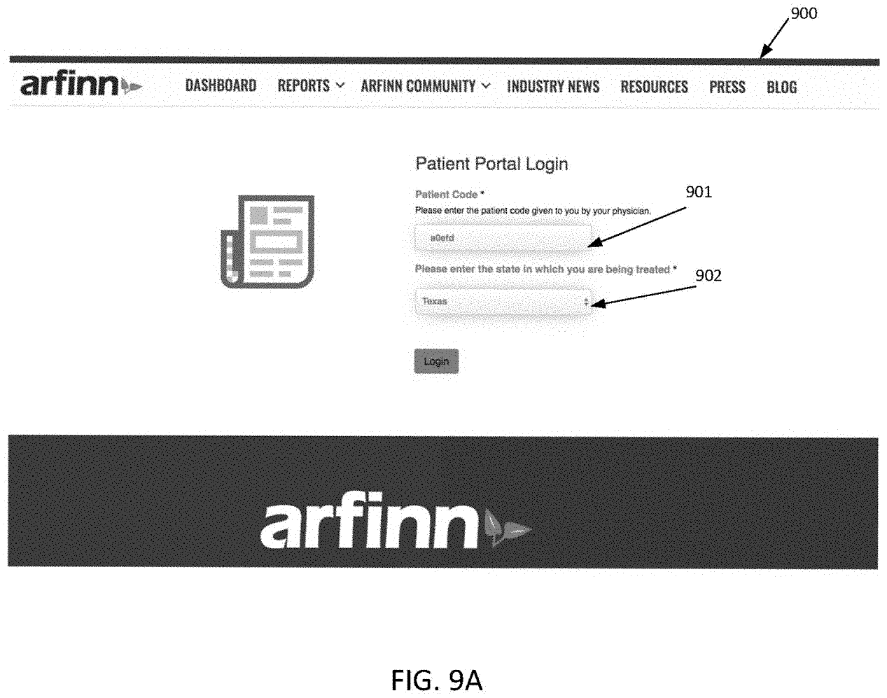

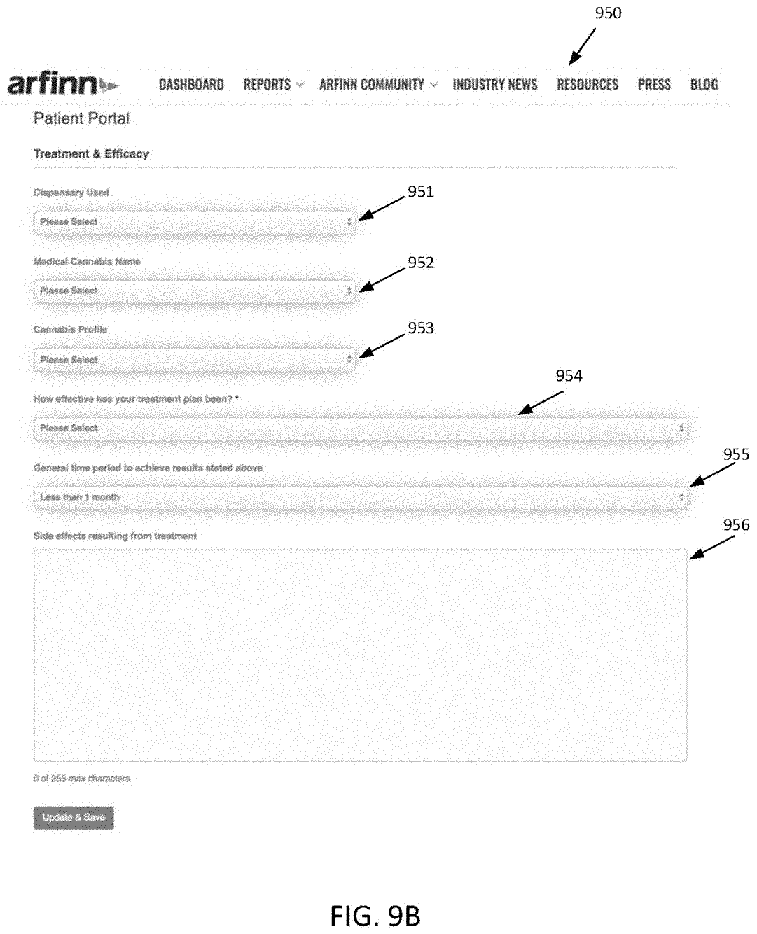

FIG. 4 illustrates an exemplary cannabis treatment platform accessible through a patient portal according to some embodiments of the present invention. The patient 150 receives the electronic message sent by the platform 102 that contains the access code and optionally the notification of the availability of the cannabis treatment prescription (401). After obtaining the cannabis products) from a dispensary 140 (405), the patient 150, via the patient portal 112, logs into the, platform 102 using the access code, and the platform 102 authenticates the patient's access using the access code (406). FIG. 9A illustrates an example patient portal login interface 900. The patient enters the access code in the patient code field 901 and selects the state in which the patient is being treated 902. The state provides a secondary piece of information to verify the patient 150. The patient 150, through a web form on the patient portal 112, enters patient treatment data (407), such as their cannabis treatment outcomes, side effects, efficacy, or treatment questions. FIG. 9B illustrates an example of a patient portal input web form. The platform 102 receives the patient treatment data, which includes the dispensary used 951, the name of the medical cannabis 952 actually used, the cannabis profile 953 for the actual cannabis product used, the patient efficacy rating 954 (i.e., the patient's rating of how effective the treatment has been), the time period in which the inputted results were achieved 955, and a description of any side effects resulting from the treatment 956. In some embodiments, the cannabis profile is automatically extracted from a source internal or external to the platform 102 based on an identity of the actual cannabis product used. Other patient treatment data that may be requested include, but are not limited to: intake method; results; dosage; and side effects. The platform 102 stores the patient treatment data in the patient record associated with the patient 150 (408). This data then becomes part of the corpus of data used in further analyses and report generation, as described above. The platform 102 then, sends an electronic message to the physician 160 with a notification of the availability of the patient cannabis treatment data (409).

When the electronic message sent per block 211 of FIG. 2 also includes the notification of availability of the cannabis treatment prescription, the patient 150, via the patient portal 112, logs into the platform 102 using the access code, and the platform 102 authenticates the patient's access using the access code (402). The patient 150 requests the cannabis treatment prescription via the patient portal 112. The platform 102 receives the request for the cannabis treatment prescription from the patient 150 (403), and in response, retrieves and outputs the cannabis treatment prescription stored in the patient record associated with the patient 150 (404). The patient 150 then obtains cannabis product(s) from, a cannabis dispensary 140 (405). At some later point in time, the patient 150 logs into the platform 102 again via the patient portal 112. The platform 102 again authenticates the patient's access to the platform 102 using the access code (406), and the patient 150 inputs the cannabis treatment data per blocks 407-409), as described above.

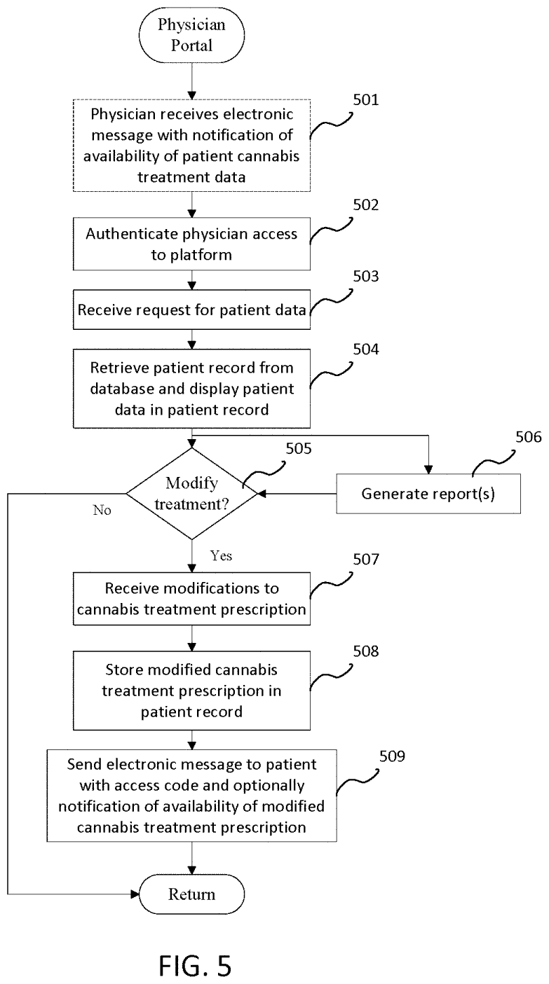

Referring to FIG. 5, when the physician 160 receives the electronic message with the notification of the availability of the patient, cannabis treatment data (501), the physician logs into the platform 102 via the physician portal 122. The platform 102 authenticates the physician access (502). The physician 160, via the physician portal 122 requests to view the patient data for the patient 150. The platform 102 receives the request for the patient data (503), and in response, retrieves the patient record from the database 170 and displays the patient data stored in the patient record (504). The physician 160 is then given an opportunity to modify the cannabis treatment prescription for the patient 150. Based on the patient's inputted treatment data, the physician 160 may decide to modify the cannabis treatment prescription, such as the cannabis type, delivery method, or dosage, in order to reduce the side effects or to increase the efficacy. Optionally, the physician 160 may generate one or more reports (506), as described above with reference to FIGS. 2 and 3, prior to making modifications. When the platform 102 receives modifications to the patient's cannabis treatment prescription (507), the platform 102 stores the modified cannabis treatment prescription in the patient record associated with the patient 150 (508). The platform 102 sends another electronic message to the patient 150 with the access code and optionally a notification of the availability of the modified cannabis treatment prescription (509). The patient 150 can then access the modified cannabis treatment prescription and/or input updates to the patient cannabis treatment data, as described above with reference to FIG. 4,

The cannabis treatment platform 102 according to embodiments of the present invention provide physicians and patient with the ability to view and contribute to information on cannabis treatments. The physician is thus able to base the prescribed cannabis treatment on actual treatment data.

The intent of the physician's prescription of the cannabis treatment is for the patient 150 to share the cannabis treatment prescription with the dispensary 140 and for the dispensary 140 makes recommendations on the cannabis products accordingly. However, unlike traditional medicine, the dispensary 140 is not required to dispense according to the physician's cannabis treatment prescription. Instead the actual cannabis product purchased by the patient 150 is a decision made with the dispensary. Further, in some jurisdictions, the state can regulate factors, such as delivery mode and daily dosage. 140 without involvement of the physician 160. For this reason, an electronic communication channel between the physician 160 and the patient 150 does not currently exist where the physician 160 can monitor the actual cannabis product, used and/or the progress of the patient's cannabis treatment in a meaningful way. The platform 102 solves this problem by implementing a communication channel over the network 130 between the physician 160 and the cannabis patient 150.

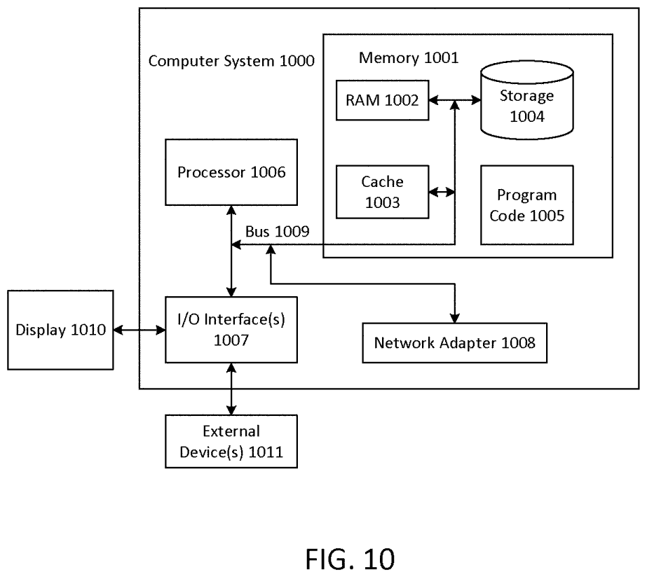

FIG. 10 illustrates a computing system, one or more of which may be used to implement the server 101, the patient client device 110, and/or the physician client device 120, according to various embodiments of the present invention. The computing system 1000 is operationally coupled to a processor or processing units 1006, a memory 1001, and a bus 1009 that couples various system components, including the memory 1001 to the processor 1006. The bus 1009 represents one or more of any of several types of bus structure, including a memory bus or memory controller, a peripheral bus, an accelerated graphics port, and a processor or local bus using any of a variety of bus architectures. The memory 1001 may include computer readable media in the form of volatile memory, such as random access memory (RAM) 1002 or cache memory 1003, or non-volatile storage media 1004. The memory 1001 may include at least one program product having a set of at least one program code module 1005 that are configured to carry out the functions of embodiment of the present invention when executed by the processor 1006. The computer system 1000 may also communicate with one or more external devices 1011, such as a display 1010, via I/O interfaces 1007. The computing system 1000 may communicate with one or, more networks via network adapter 1008.

The present invention can take the form of an entirely hardware embodiment, an entirely software embodiment or an embodiment containing both hardware and software elements. In a preferred embodiment, the present>invention is implemented in software, which includes but is not limited to firmware, resident software, microcode, etc.

Furthermore, the present invention can take the form of a computer program product accessible from a computer usable or computer readable storage medium providing program code for use by or in connection with a computer or any instruction execution system. For the purposes of this description, a computer usable or computer readable storage medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W) and DVD. A computer readable storage medium, as used herein, is not to be construed as being transitory signals per se, such as radio waves or other freely propagating electromagnetic waves electromagnetic waves propagating through a waveguide or other transmission media (e.g., light pulses passing through a fiber-optic cable), or electrical signals transmitted through a wire.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but, not limited to keyboards, displays, point devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

The flowchart and block diagrams in the Figures illustrate the architecture, functionality, and operation of possible implementations of systems, methods and computer program products according to various embodiments of the present invention. In this regard, each block in the flowchart or block diagrams may represent a module segment or portion of code, which comprises one or more executable instructions for implementing the specified local function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. A method for providing networking communication channels between a physician and a patient for cannabis treatment and result data, comprising:
(a) receiving, by a computing platform from a first client device of the physician, input of a medical condition for the patient
(b) finding, by the computing platform, a set of patient records in a database matching the medical condition;
(c) calculating, by the computing platform, an efficacy rating for the set of patient records;
(d) generating, by the computing platform, for display at the first client device, one or more reports of cannabis treatment profiles in the set of patient records and corresponding efficacy ratings;
(e) receiving, by the computing platform from the first client device, a cannabis treatment prescription for the patient;
(f) storing, by the computing platform, the cannabis treatment prescription in a patient record associated with the patient;
(g) generating, by the computing platform, a unique access code and associating the unique access code with the patient; and
(h) sending, by the computing platform, a first electronic message to the patient comprising the unique access code.

2. The method of claim 1, wherein the receiving (e) comprises:
(e1) receiving, by the computing, platform from the first client device, a selection of a given cannabis treatment, profile in the one or more reports as the cannabis treatment prescription for the patient.

3. The method of claim 1, wherein the receiving (e) comprises:
(e1) receiving, by the computing, platform from the first client device, one or more modifications to a given cannabis treatment profile in the one or more reports; and
(e2) receiving, by the computing platform from the first client device, a selection of the modified given cannabis treatment profile as the cannabis treatment prescription for the patient.

4. The method of claim 1, wherein the generating (d) comprises:
(d1) determining, by the computing platform, a population size with the medical condition;
(d2) calculating, by the computing platform, a sample size of the population that meets a predetermined confidence level;
(d3) determining, by the computing platform, whether the set of patient records is greater than or equal to the sample size;
(d4) when the set of patient, records is not greater than or equal to the sample size, using, by the computing platform, the set of patient records as sample patient records;
(d5) when the set of patient records is greater than or equal to the sample size, selecting, by the computing platform, a subset of the set of patient records as the sample patient records according to the sample size;
(d6) calculating, by the computing platform, an average efficacy rating for the sample patient records;
(d7) creating, by the computing platform, a frequency distribution, of the efficacy ratings in the set of patient records; and
(d8) generating, by the computing platform, the one or more reports based on the average efficacy ratings or the frequency distribution of efficacy ratings.

5. The method of claim 1, wherein after the patient obtains one or more cannabis products, the method further comprises:
(i) receiving, by the computing platform from a second client device of the patient an access request comprising the unique access code;
(j) in response to the access request, authenticating using the unique access code, by the computing platform, access to the computing platform by the second client device;
(k) receiving, by the computing platform from the second client device, input, of cannabis treatment data for the patient comprising a cannabis profile of the one or more cannabis products and a patient efficacy rating;
(l) storing, by the computing platform, the cannabis treatment data in the patient record associated with the patient; and
(m) sending, by the computing platform, a second electronic message to the physician comprising a notification of an availability of the cannabis treatment data input by the patient.

6. The method of claim 5, further comprising:
(o) receiving, by the computing platform from the first client device, a request for the cannabis treatment data input by the patient; and
(p) in response to the request for the cannabis treatment data input by the patient, retrieving, by the computing platform, the patient record associated with the patient from the database.

7. The method of claim 6, further comprising:
(q) receiving, by the computing platform from the first client device, one or more modifications to the cannabis treatment prescription for the patient;
(r) storing, by the computing platform, the modified cannabis treatment prescription in the patient record associated with the patient; and
(s) sending, by the computing platform, a fourth electronic message to the patient comprising the access code.

8. The method of claim 7, wherein the fourth electronic message further comprises a notification of an availability of the modified cannabis treatment prescription for the patient.

9. The method of claim 1, wherein the first electronic message further comprises a notification of an availability of the cannabis treatment prescription for the patient.

10. A non-transitory computer readable medium comprising computer readable program code embodied therein, wherein when executed by a processor causes the processor to:
(a) receive, from a first client device of a physician, input of a medical, condition for a patient;
(b) find a set of patient records in a database matching the medical condition;
(c) calculate an efficacy rating for the set of patient records;
(d) generate, for display at the first client device, one or more reports of cannabis treatment profiles in the set of patient records and, corresponding efficacy ratings;
(e) receive, from the first client device, a cannabis treatment prescription for the patient;
(f) store the cannabis treatment prescription in a patient record associated with the patient;
(g) generate a unique access code and associating the unique access code with the patient; and
(h) send a first electronic message to the patient comprising the unique access code.

11. The medium of claim 10, wherein the receive (e) comprises:
(e1) receive, from the first client device, one or more modifications to a given cannabis treatment profile in the one or more reports; and
(e2) receive, from the first client device, a selection of the modified given cannabis treatment profile as the cannabis treatment prescription for the patient.

12. The medium of claim 10, wherein after the patient obtains one or more cannabis products, the processor is further caused, to:
(i) receive, from a second client device of the patient, an access request comprising the unique access code;
(j) in response to the access request, authenticate using the unique access code access to the computing platform by the second client device;
(l) receive, from the second client device, input of cannabis treatment data for the patient comprising a cannabis profile of the one or more cannabis products and a patient efficacy rating;
(m) store the cannabis treatment data in the patient record associated with the patient; and
(n) send a second electronic message to the physician comprising a notification of an availability of the cannabis treatment data input by the patient.

13. The medium of claim 12, wherein the processor is further caused to:
(o) receive, from the first client device, a request for the cannabis treatment data input by the patient; and
(p) in response to the request for the cannabis treatment data input by the patient, retrieve the patient record associated with the patient from the database.

14. The medium of claim 13, wherein the processor is further caused to:
(q) receive, from the first client device, one or more modifications to the cannabis treatment prescription for the patient;
(r) store the modified cannabis treatment prescription in the patient record associated with the patient; and
(s) send a fourth electronic message to the patient comprising the access code.

15. A system, comprising:
a computer readable storage medium having computer readable program code embodied therewith, the computer readable program code configured to:
(a) receive, from a first client device of a physician, input of a medical condition for a patient;
(b) find a set of patient records in a database matching the medical condition;
(c) calculate an efficacy rating for the set of patient records;
(d) generate, for display at the first client device, one or more reports of cannabis treatment profiles in the set of patient records and corresponding efficacy ratings;
(e) receive, from the first client device, a cannabis treatment prescription for the patient;
(f) store the cannabis treatment prescription in a patient record associated with the patient;
(g) generate a unique access code and associating the unique access code with the patient; and
(h) send a first electronic message to the patient comprising the unique access code.

16. The system of claim 15, wherein the receive (e) comprises:
(e1l) receive, from the first client device, one or more modifications to a given cannabis treatment profile in the one or more reports; and
(e2) receive, from the first client device, a selection of the modified given cannabis treatment profile as the cannabis treatment prescription for the patient.

17. The system of claim 15, wherein after the patient obtains one or more, cannabis products, the processor is further caused to:
(i) receive, from a second client device of the patient, an access request comprising the unique access code;
(j) in response to the access request, authenticate using the unique access code access to the computing platform by the second client device;
(k) receive, from the second client device, input of cannabis treatment data for the patient comprising a cannabis profile of the one or more cannabis products and a patient efficacy rating;
(l) store the cannabis treatment data in the patient record associated with the patient; and
(m) send a second electronic message to the physician comprising a notification of an availability of the cannabis treatment data input by the patient.

18. The system of claim 17, wherein the processor is further caused to:
(o) receive, from the first client device, a request for the cannabis treatment data input by the patient and
(p) in response to the request for the cannabis treatment data input by the patient, retrieve the patient record associated with the patient from the database.

19. The system of claim 18, wherein the processor is further caused to:
(q) receive, from the first client device, one or more modifications to the cannabis treatment prescription for the patient;
(r) store the modified cannabis treatment prescription in the patient record associated with the patient; and
(s) send a fourth electronic message to the patient comprising the access code.
